(19) United States
(12) Patent Application Publication
(10) Pub. No.: US 2023/0074885 A1
(43) Pub. Date: Mar. 9, 2023

(54) BORTEZOMIB-LOADED NANOPARTICLES

(71) Applicant: The Johns Hopkins University, Baltimore, MD (US)

(72) Inventors: Florin M. Selaru, Catonsville, MD (US); Hai-Quan Mao, Baltimore, MD (US); Ling Li, Baltimore, MD (US); Yang Zhou, Baltimore, MD (US); Zhiyu He, Baltimore, MD (US); Kuntao Chen, Philadelphia, PA (US)

(21) Appl. No.: 17/797,053

(22) PCT Filed: Feb. 5, 2021
(86) PCT No.: PCT/US2021/016849

§ 371 Date: Aug. 2, 2022

Related U.S. Application Data
(60) Provisional application No. 62/970,421, filed on Feb. 5, 2020.

(51) Int. Cl. A61K 47/64; A61K 9/51; A61K 47/54; A61K 45/06; A61P 35/00

(52) U.S. Cl. CPC A61K 47/643; A61K 9/5153; A61K 47/542; A61K 45/06; A61P 35/00

(57) ABSTRACT

The presently disclosed subject matter provides nanoparticles comprising bortezomib encapsulated in a non-water-soluble polymer matrix in a form of a bortezomib-tannic acid complex; methods for preparing the nanoparticle; and use of the nanoparticles for treating liver cancer.

### BACKGROUND

The incidence of liver cancer in 2019 in the United States is estimated to be about 42,000, and in the world 840,000. There is currently no curative chemotherapy strategy on the market, however, for un-resectable advanced liver cancer. Some experts have hypothesized that hepatocellular cancer (HCC) and cholangiocarcinoma (CCA) are “not druggable.” Llovet et al., 2016. Thus, there is a need in the art for delivering therapeutic agents to liver cancers.

### SUMMARY

In some aspects, the presently disclosed subject matter provides a nanoparticle comprising bortezomib encapsulated in a non-water-soluble polymer matrix in a form of a bortezomib-tannic acid complex.

In certain aspects, the bortezomib-tannic acid complex is bonded and stabilized to one or more proteins or peptides via hydrogen bond formation. In particular aspects, the weight percentage of the one or more protein or peptides is the range from about 5 w/w % to about 20 w/w %. In particular aspects, the one or more proteins or peptides has a molecular weight in the range of about 1 kDa to about 160 kDa. In more particular aspects, the one or more proteins comprises serum albumins, such as recombinant human serum albumin, bovine serum albumin, mouse serum albumin, ovalbumin, collagen, gelatin, or protamine.

In certain aspects, the non-water-soluble polymer matrix comprises one or more biodegradable polyesters. In particular aspects, the non-water-soluble polymer matrix comprises one or more polymers selected from the group including poly(D-lactic acid) (PDLA), poly(L-lactic acid) (PLLA), poly(D,L-lactic acid) (PDLLA), poly(glycolic acid) (PGA), polycaprolactone (PCL), and their copolymers such as poly(lactic acid-co-glycolic acid) (PLGA), poly(caprolactone-co-glycolic acid) (PCLGA), and the like. In more particular aspects, the non-water-soluble polymer matrix comprises one or more block copolymers of polyester with poly(ethylene glycol) (PEG), selected from the group including poly(ethylene glycol)-b-poly(D-lactic acid) (PEG-b-PDLA), poly(ethylene glycol)-b-poly(L-lactic acid) (PEG-b-PLLA), poly(ethylene glycol)-b-poly(D,L-lactic acid) (PEG-b-PDLLA), poly(ethylene glycol)-b-poly(glycolic acid) (PEG-b-PGA), poly(ethylene glycol)-b-polycaprolactone (PEG-b-PCL), poly(ethylene glycol)-b-poly(lactic acid-co-glycolic acid) (PEG-b-PLGA), poly(ethylene glycol)-b-poly(caprolactone-co-glycolic acid) (PEG-b-PCLGA), and the like. In even more particular aspects, the non-water-soluble polymer comprises poly(ethylene glycol)-b-poly(lactic acid-co-glycolic acid) (PEG-b-PLGA).

In certain aspects, the bortezomib is released from the nanoparticle over a period of time ranging from about 2 to about 60 days in vitro.

In some aspects, the nanoparticle further comprises one or more additional chemotherapy agents.

In other aspects, the presently disclosed subject matter provides a method for making a nanoparticle, the method comprising: (a) mixing bortezomib (BTZ) and tannic acid (TA) to form a BTZ/TA complex; (b) mixing a protein with the TA/BTZ complex forming a BTZ/TA/protein complex; (c) mixing a non-water soluble polymer with the BTZ/TA/protein complex; and (d) forming a nanoparticle.

In certain aspects, the tannic acid is in an aqueous solution and has a concentration ranging from about 1 mg/mL to about 20 mg/mL. In certain aspects, the BTZ is in a solution comprising 0-10% acetonitrile/2-10% dimethyl sulfoxide/80-96% water. In certain aspects, the tannic acid and the bortezomib are mixed by simultaneously injecting tannic acid and bortezomib into a 2-inlet confined impinging jet (CIJ) mixer at a flow rate in the range of about 0.2 to about 25 mL/min to form the TA/BTZ complex.

In particular aspects, the BTZ/TA complex and the protein are mixed in an aqueous suspension by simultaneously injecting the BTZ/TA complex and the protein into a second 2-inlet CIJ mixer at a flow rate of about 0.2 to about 25 mL/min to form the protein complex.

In certain aspects, the protein comprises a serum albumin, such as recombinant human serum albumin, bovine serum albumin, mouse serum albumin, ovalbumin, collagen, gelatin, or protamine.

In certain aspects, the protein complex and the non-water-soluble polymer are mixed in DMSO/acetonitrile mixture at a volume ratio of about 0 to about 1 by simultaneously injecting the BTZ/TA/protein complex and the non-water-soluble polymer into a 3-inlet CIJ mixer at a flow rate of about 0.1 to about 25 mL/min, thereby forming the nanoparticles.

In certain aspects, the non-water-soluble polymer matrix comprises one or more biodegradable polyesters. In particular aspects, the non-water-soluble polymer matrix comprises one or more polymers selected from the group including poly(D-lactic acid) (PDLA), poly(L-lactic acid) (PLLA), poly(D,L-lactic acid) (PDLLA), poly(glycolic acid) (PGA), polycaprolactone (PCL), and their copolymers such as poly(lactic acid-co-glycolic acid) (PLGA), poly(caprolactone-co-glycolic acid) (PCLGA), and the like. In more particular aspects, the non-water-soluble polymer matrix comprises one or more block copolymers of polyester with poly(ethylene glycol) (PEG), selected from the group including poly(ethylene glycol)-b-poly(D-lactic acid) (PEG-b-PDLA), poly(ethylene glycol)-b-poly(L-lactic acid) (PEG-b-PLLA), poly(ethylene glycol)-b-poly(D,L-lactic acid) (PEG-b-PDLLA), poly(ethylene glycol)-b-poly(glycolic acid) (PEG-b-PGA), poly(ethylene glycol)-b-polycaprolactone (PEG-b-PCL), poly(ethylene glycol)-b-poly(lactic acid-co-glycolic acid) (PEG-b-PLGA), poly(ethylene glycol)-b-poly(caprolactone-co-glycolic acid) (PEG-b-PCLGA), and the like. In even more particular aspects, the non-water-soluble polymer comprises poly(ethylene glycol)-b-poly(lactic acid-co-glycolic acid) (PEG-b-PLGA).

In other aspects, the presently disclosed subject matter provides a method for treating liver cancer in a subject in need of treatment thereof, the method comprising delivering one or more of the presently disclosed nanoparticles to the subject by intratumor injection to treat the liver cancer.

In certain aspects, the intratumor injection is in an artery forming an intratumor injection tract and further comprises the step of blocking off the artery(ies) that feed the liver cancer after the delivery of the nanoparticle. In particular aspects, the blocking occurs by transarterial embolization.

In certain aspects, the method further comprises plugging an intratumor injection tract. In certain aspects, the one or more nanoparticles are delivered by catheter-based intra-tumoral intra-vascular delivery. In particular aspects, the catheter-based intratumoral intra-vascular delivery is followed by an embolization blockage to achieve a local retention and release of bortezomib.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one figure executed in color. Copies of this patent or patent application publication with color figures will be provided by the Office upon request and payment of the necessary fee.

Having thus described the presently disclosed subject matter in general terms, reference will now be made to the accompanying Figures, which are not necessarily drawn to scale, and wherein:

FIG. 1 is a schematic diagram of the sequential Flash Nanocomplexation (FNC)/Flash Nanoprecipitation (FNP) process;

FIG. 2A and FIG. 2B show formulation screening of TA-BTZ and TA-BTZ-OVA complex. (FIG. 2A) Size distribution of TA-BTZ complex under different flow rate. (FIG. 2B) Size distribution of TA-BTZ-OVA complex under different flow rate;

FIG. 3A, FIG. 3B, FIG. 3C, and FIG. 3D show characterization of BTZ-loaded nanoparticles. (FIG. 3A) Encapsulation efficiency of BTZ; (FIG. 3B) Size distribution of BTZ-loaded nanoparticles; (FIG. 3C) Surface charge and size change of nanoparticle along each step. (FIG. 3D) Transmission electron microscopy imaging of BTZ-loaded nanoparticles;

FIG. 4 is an in vitro release study of BTZ-loaded nanoparticles. BTZ-TA complex and BTZ-TA-OVA complex without PEG-b-PLGA coating; NP-0 refers to TA/BTZ complex coated directly with PEG-b-PLGA; NP-1, NP-2, NP-3 refer to BTZ/TA/OVA/PEG-PLGA nanoparticle prepared under different PEG-PLGA concentrations. The results are presented as mean±S.D. (n=3);

FIG. 5 is a bioactivity evaluation of BTZ-loaded nanoparticles by measuring MDA cell viability in 4 h upon free BTZ, BTZ/TA complex and BTZ-loaded nanoparticles dosing through MTT assay. ***p<0.001 compared to PBS control. The results are presented as mean±S.D. (n=3);

FIG. 6 is an in vivo treatment of a patient derived xenograft (PDX) mouse model of liver cancer. 100 μL BTZ-loaded nanoparticles were directly injected into the tumor at day 0, 100 μL blank polymer was utilized as negative control. The tumor sizes of treatment group and negative control group are measured every other day. The results demonstrated that while tumors treated with negative control (blank polymer) continue to grow, tumors treated with BTZ-loaded nanoparticles diminish in size;

FIG. 7A and FIG. 7B show the biodistribution of fluorescently-labeled nanoparticles after intra-tumor injection demonstrating that the nanoparticles are retained in the tumor mass. (FIG. 7A) In vivo fluorescence imaging of mice at different time points (0-240 h) after a single intra-tumor injection of Cy 7.5-labelled nanoparticles. (FIG. 7B) Quantification of fluorescence intensity of the Cy 7.5-labelled nanoparticle at injection site. The results are presented as mean±S.D. (n=³);

FIG. 8A, FIG. 8B, FIG. 8C, FIG. 8D, and FIG. 8E show the effect of formulation parameters for nanoparticles on BTZ release rate and duration. BTZ NPs prepared with the following conditions: (FIG. 8A) pH=5, flow rate=1 mL/min, PEG5 KDa-b-PLGA20 KDa; (FIG. 8B) pH=7, flow rate=1 mL/min, PEG5 KDa-b-PLGA20 KDa (FIG. 8C) pH=9, flow rate=1 mL/min, PEG5 KDa-b-PLGA20 KDa; (FIG. 8D) pH=5, flow rate=10 mL/min, PEG5 KDa-b-PLGA20 KDa; (FIG. 8E) pH=9, flow rate=10 mL/min, PEG5 KDa-b-PLGA20 KDa; (FIG. 8A) pH=9, flow rate=10 mL/min, PEG5 KDa-b-PLGA45 KDa;

FIG. 9 shows the release profiles of four selected BTZ NP formulations showing the effect of pH and flow rate during nanoparticle assembly. NP #1 was prepared at low pH (pH 5), low flow rate (1 mL/min) and without protein co-encapsulation; NP #2 was prepared at low pH (pH 5), low flow rate (1 mL/min) and with ovalbumin co-encapsulation; NP #3 was prepared at high pH (pH 9), low flow rate (1 mL/min) and with ovalbumin co-encapsulation; NP #4 was prepared at high pH (pH 9), high flow rate (10 mL/min) and with ovalbumin co-encapsulation. All these formulations were prepared with PEG5 KDa-b-PLGA20 KDa. NP #2 was termed as the 1-week NP formulation, and NP #4 was termed as the 1-month NP formulation. Non-linear and linear curve fitting was used to estimate the releasing rates of BTZ in each NP formulation; and the results were listed;

FIG. 10A, FIG. 10B, and FIG. 10C show the characterization of the 1-week formulation and the 1-month formulation. (FIG. 10A) Size distribution of both 1-week formulation and 1-month formulation. Transmission electron microscopy imaging of (FIG. 10B) 1-week formulation and (FIG. 10C) 1-month formulation of the BTZ NPs; and

FIG. 11A, FIG. 11B, and FIG. 11C show the reproducibility and scalability of the 1-month formulation. (FIG. 11A) Size distribution of multiple batches of the 1-month formulation of BTZ NPs. (FIG. 11B) Size distribution and (FIG. 11C) surface charge of the aliquots of samples from a single batch of the 1-month BTZ NP formulation.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figure. Therefore, it is to be understood that the presently disclosed subject matter is not to be limited to the specific embodiments disclosed and that modifications and other embodiments are intended to be included within the scope of the appended claims.

Thirty-seven primary patient-derived cancer organoid (PDO) cultures have been established from patients with liver cancers (including hepatocellular cancer and cholangiocarcinoma). Food and Drug Administration (FDA)-approved cancer drugs (irrespective of FDA indication) were tested across all 37 PDO cultures. Bortezomib (BTZ), a proteasome inhibitor that is FDA approved for multiple myeloma and some forms of chronic myelogenous leukemia (CML), was found to be highly effective across all tested PDO lines. Further, BTZ was found to be more effective than any of the currently approved FDA drugs for unresectable liver cancers across all 37 PDO lines. It also has been shown that BTZ is effective in a patient derived xenograft (PDX) model of liver cancer. PDX models are thought to have a positive predictive value in the high 90's for efficacy of the tested drug in patients.

The narrow therapeutic index (due to the high toxicity) of BTZ, however, prevents its future translational development for liver cancers. For example, at higher systemic doses, BTZ can induce liver dysfunction. Accordingly, the presently disclosed subject matter provides an extended-release formulation of BTZ that delivers locally in the tumor (assuring higher doses of BTZ locally with less systemic exposure), over an extended period (to prevent the need for frequent drug administration).

Currently, common strategies for the delivery of BTZ include the application of polymer, mesoporous silica, graphene oxide and liposomal nanoparticle systems. Shen et al., 2014; Ashley et al., 2014; Shen et al., 2015; Hu et al., 2018. Few of these strategies, however, have focused on the sustained release formulation of BTZ. Therefore, it is of great significance to develop a formulation that can be translated into clinical application in future use. There is report using PLGA as carrier to achieve an extended release of BTZ, yet the complicated emulsion method compromised the stability and reproducibility of the nanoparticle preparation and thus hindered its translational potential. Shen et al., 2015.

In contrast, the presently disclosed subject matter provides an affinity-enhanced BTZ sustained release nanoparticle formulation with facile preparation process using their newly developed sequential Flash Nanocomplexation/Flash Nanoprecipitation technique. A quaternary nanoparticle structure combining a ternary complex of tannic acid (TA), BTZ and ovalbumin (OVA) as core with coating of PEG-PLGA polymer matrix was constructed to successfully achieve a sustained release of BTZ for 5 days. It has previously been demonstrated that tannic acid (TA) is a suitable carrier agent in a protein sustained-release nanoparticle system owing to its versatile structure. He et al., 2019; He et al., 2020.

Similar to its binding with protein, the polyphenol structure of TA provides various binding potentials towards BTZ, including π-π stacking, hydrophobic interaction and hydrogen bonding, Hu et al., 2018, Jin et al., 2015; Le et al., 2018, which makes it an ideal choice as an extra affinity provider for BTZ in the nanoparticle system. The addition of OVA as excipient serves to lower the highly negatively charged complex surface and increase the hydrophobicity thus helps with the PEG-PLGA coating. The sequential flash nanocomplexation (FNC)/flash nanoprecipitation (FNP) platform, which can achieve a controlled nanoparticle assembly and release behavior by manipulating formulation parameters, such as mixing speed, pH or concentration, avoided cumbersome operations typically used in the emulsion methods and held great clinical translation potential in terms of particle size uniformity, reproducibility and high scalability.

Nanoparticle Assembly through Flash nanocomplexation (FNC)/Flash nanoprecipitation (FNP).

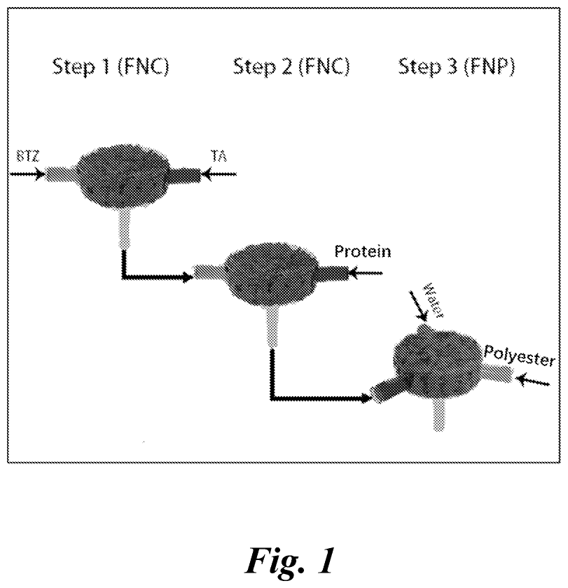

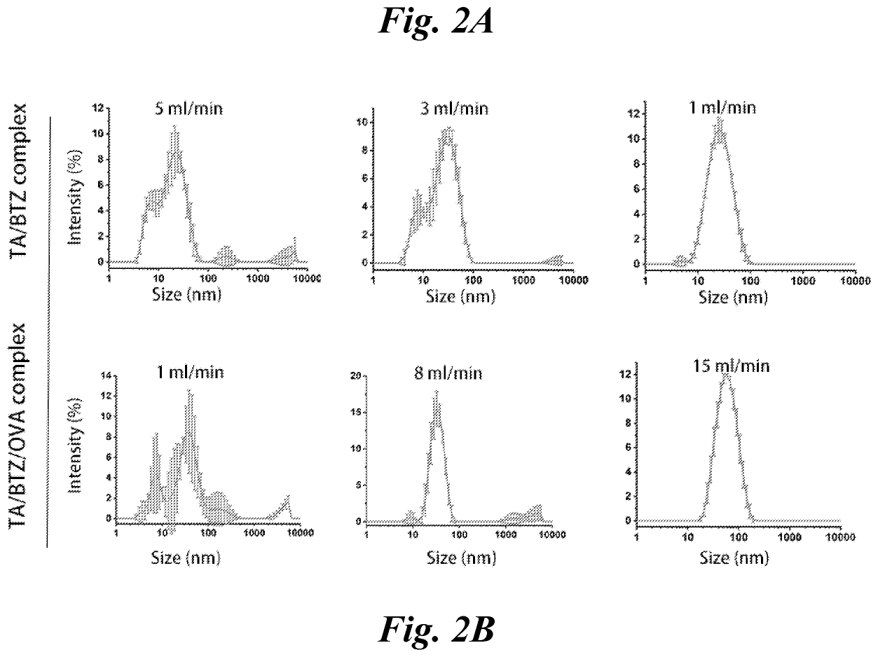

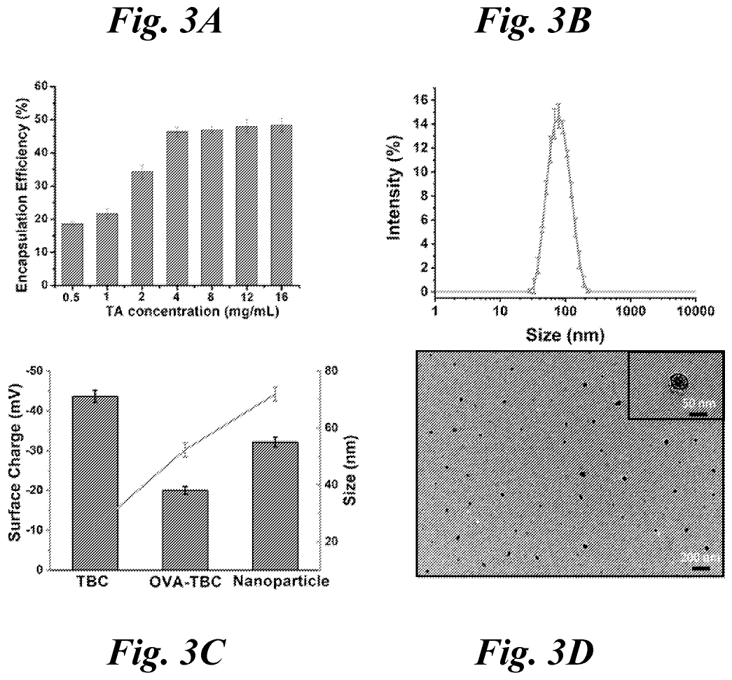

The nanoparticle was assembled by a sequential FNC/FNP process as shown in FIG. 1, in which the first step is to generate a TA-BTZ complex (TBC). It was found that the uniformity of TBC is closely associated with flow rate as higher mixing rate will result in poor size distribution (FIG. 2A). Compared to the flow rate to form TBC (1 mL/min), however, the formation of OVA-TA-BTZ complexes required a relatively high flow rate (15 mL/min) to ensure a uniform nanocomplex (FIG. 2B), which necessitated the two-step FNC process instead of a one-step ternary mixing. The encapsulation efficiency of BTZ increased with higher TA concentration, but reached a plateau at around 50% when TA/BTZ mass ratio reached 4 to 1 (FIG. 3A), therefore TA 4 mg/mL was fixed in the following experiment.

As shown in FIG. 3B and FIG. 3C, there is an increment of particle size along the addition of each component, indicating the successful construction of the BTZ-loaded nanoparticle in a stepwise assembly manner. Moreover, the high negative surface charge (−44.2±0.3 mV) of BTZ-TA complex, which is unfavorable for the coating of PEG-PLGA, was decreased to −20.8 mV after OVA coating. In such a way, it was thought that OVA could act as an excipient to enhance the compatibility between BTZ-TA complex and PEG-PLGA by decreasing surface charge and increasing hydrophobicity, thus incurring the further coating of outer polymeric matrix. The core-shell structure was revealed by the TEM imaging shown in FIG. 3D. In vitro release study of the BTZ-loaded nanoparticles.

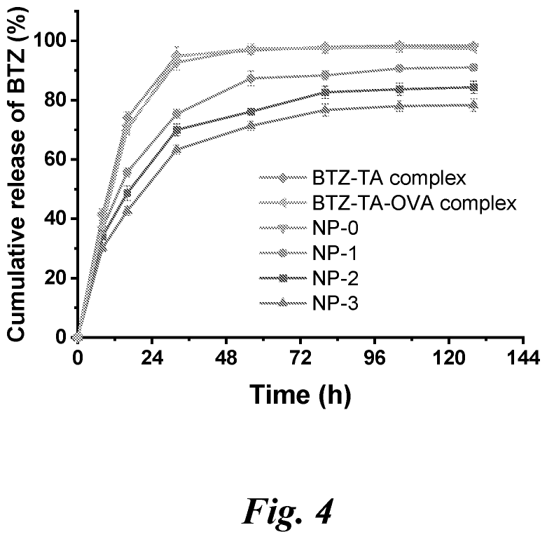

The release behavior of different formulations in PBS (pH=7.4) were compared. As shown in FIG. 4, NP-0, TA-BTZ and TA-BTZ-OVA complex all showed a burst release of BTZ up to 100% in 1 day, yet NP-1, 2, 3 can achieved a release duration around 5 days, which validated two essential assumptions: (1) A extended release of BTZ cannot be achieved without the coating of polymer matrix, which serves as an important diffusion barrier that slow down the release of BTZ; (2) TA-BTZ complex alone is not compatible enough to be coated with polymer matrix, which necessitates the incorporation of protein (OVA) as a surfactant-acting excipient. Furthermore, the release rate can be tuned down by increasing polyester concentration in the preparation process, which provides the possibility to continue to optimize the formulation and increase the duration of sustained release.

### Assessment of Cytotoxicity Using MTT Assay.

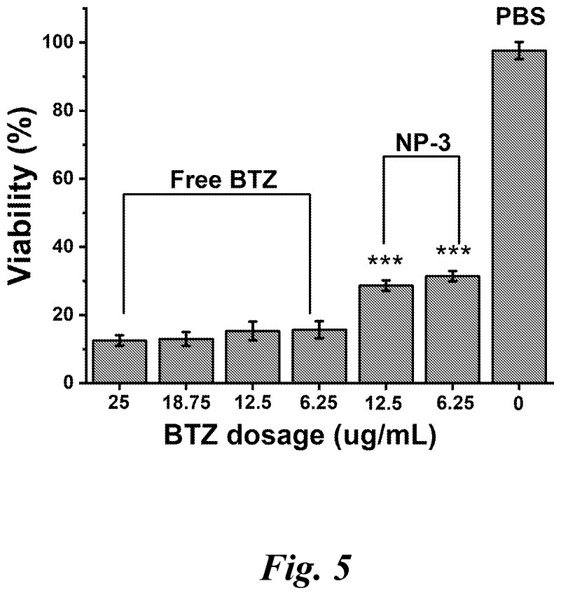

The tumor-killing effect of BTZ-loaded nanoparticles was first evaluated by observing the viability of MDA cell dosed with different formulations of BTZ through MTT assay. As shown in FIG. 5, although cell viability in the nanoparticle group is not as low as that of the same dose of free BTZ group, which is due to the sustained release behavior of BTZ from the nanoparticles, the BTZ-loaded nanoparticles still exhibited a substantial tumor killing effect (approximately 70%) in comparison with the negative control. This phenomenon suggested that the released BTZ from nanoparticle holds well preserved bioactivity, which is an important guarantee for the long-lasting tumor killing ability of the nanoparticle in vivo.

### Inhibition of Tumor Growth in PDX Model.

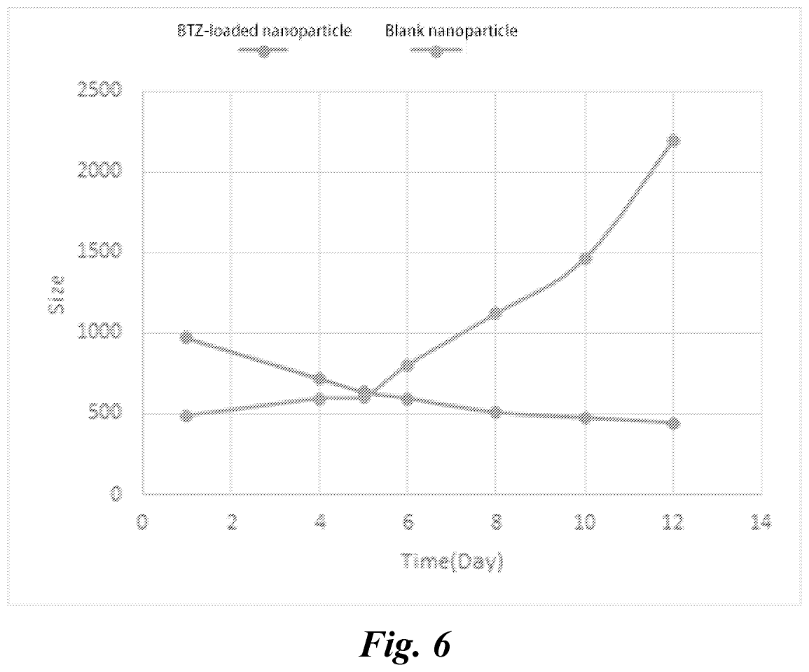

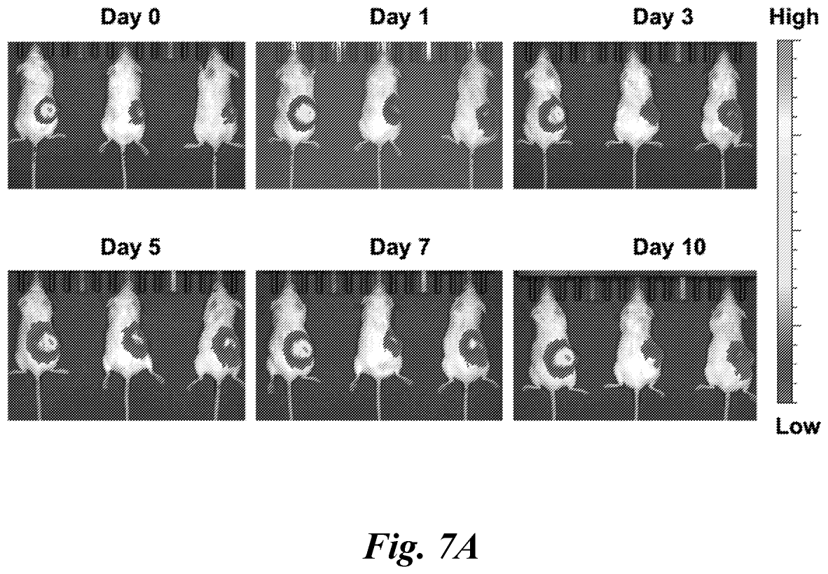

A patient derived xenograft (PDX) model was established from a human primary HCC. Mice treated with a high dose (0.8 mg/kg) of free BTZ died 1 day after injection due to the toxicity of the immediate release, high dose BTZ. Mice treated with blank polymer showed a significant tumor size growth to almost 5 times as original ones in 12 days (FIG. 6). In contrast, nanoparticles loaded with a high dose of BTZ (2.2 mg/kg) that is released in a sustained fashion successfully avoided systemic toxicity while delivering BTZ locally to the tumors and significantly inhibiting the tumor growth compared to blank nanoparticle treatment group. Without wishing to be bound to any one particular theory, it was thought that the tumor inhibition effect was likely due to a combination of burst release, which permits an initial dose of the drug to over the therapeutic window (delivered locally), and followed by a lower concentration of sustained release to maintain the BTZ dose in the tumor tissue.

In summary, the extended release formulation of BTZ demonstrated a high efficacy in a PDX model of liver cancer. PDX models are known to have a high positive predictive value (in the 90's) for the effect of the drug in the patient from whom the PDX was derived.

Persistence of Cy7.5 as a Drug Surrogate following intra-tumor injection of nanoparticles.

The therapeutic effect of BTZ-loaded nanoparticles relies on the property that after injection, the nanoparticles will be retained in the tumor and gradually release BTZ, rather than be rapidly cleared by the blood stream. Due to a previously described phenomenon of enhanced permeability and retention (EPR) effect, nanosized agents tend to be enrich and retained in the tumor tissue. Nakamura et al., 2016.

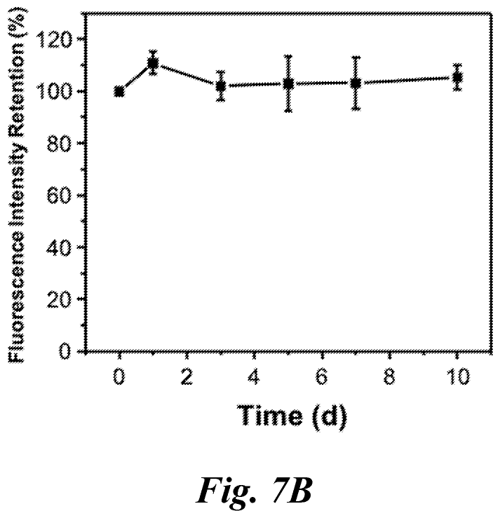

To test the presently disclosed formulated BTZ for its ability to be retained in the tumor mass, the release of fluorescently dye-labeled nanoparticles was monitored after intra-tumor injection for 360 h. As shown in FIG. 7A, the fluorescence intensity was maintained at a high level throughout the experiment, indicating an excellent retention effect of the presently disclosed nanoparticles in the tumor mass. This finding was further confirmed by the semi-quantitative analysis shown in FIG. 7B, which ensured that the BTZ could be slowed released within the tumor tissue during the treatment period considering the retention time (greater than 350 h) is much longer than the release time (approximately 120 h).

The nanoparticle formulation of BTZ described herein, in some embodiments, can be delivered via direct delivery into the tumor. HCCs have only one or a few arteries that feed the tumor, therefore, for this particular tumor, it is feasible using current clinical protocols to catheterize the main feeding arteries and deliver drugs locally. In addition, after drug delivery, the artery can be embolized to decrease further blood flow through that arterial branch and by extension diminish the likelihood of BTZ-nanoparticle being washed away.

For liver tumors outside of HCC (cholangiocarcinoma or metastatic cancers to the liver), the nanoparticle-BTZ formulation can be delivered by transcutaneous (image guided) injection into the tumor. The injection tract also could be plugged after the injection to minimize drug elimination or tumor seeding along the injection tract. It also is contemplated that the nanoparticle-BTZ formulation could be further developed for subcutaneous/depot injection. The BTZ-extended release formulation also can be administered in a subcutaneous or similar fashion for systemic extended release. The systemic extended release can be beneficial in tumors that have multiple masses in the liver (intra liver metastasis) or metastasis to other organs.

Embodiments of the disclosure concern methods and/or compositions for treating and/or preventing a liver cancer. In certain embodiments, individuals with a liver cancer, such as HCC, are treated with a nanoparticle of the present invention.

In particular, embodiments of the disclosure, a subject is given an agent for liver cancer therapy in addition to the one or more nanoparticles of the present invention. When combination therapy is employed with one or more nanoparticles of the present invention, the additional therapy may be given prior to, at the same time as, and/or subsequent to the one or more nanoparticles of the present disclosed subject matter.***

### Extending Releasing Duration of the BTZ-Loaded Nanoparticles (BTZ-NPs).

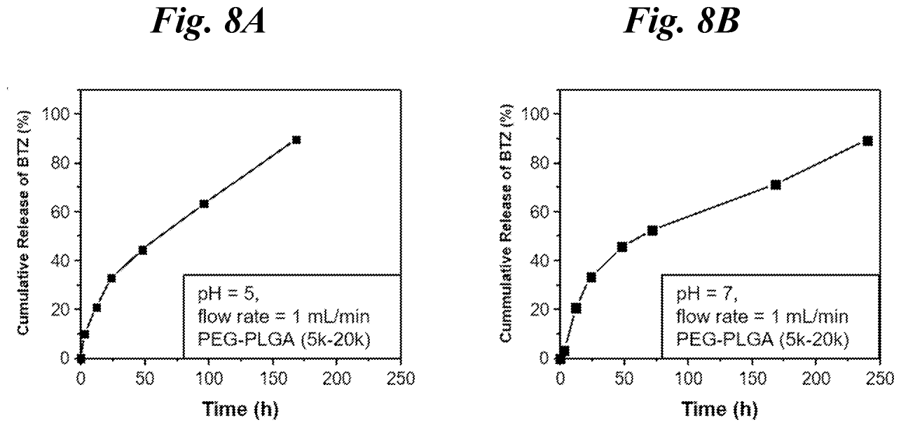

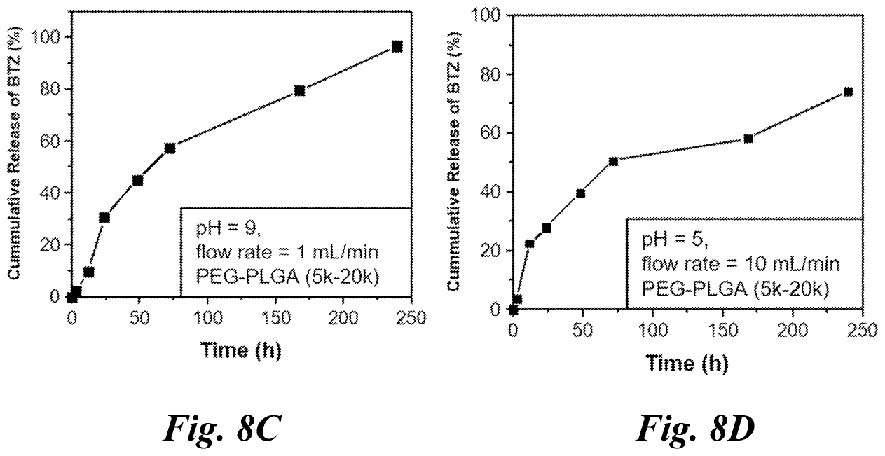

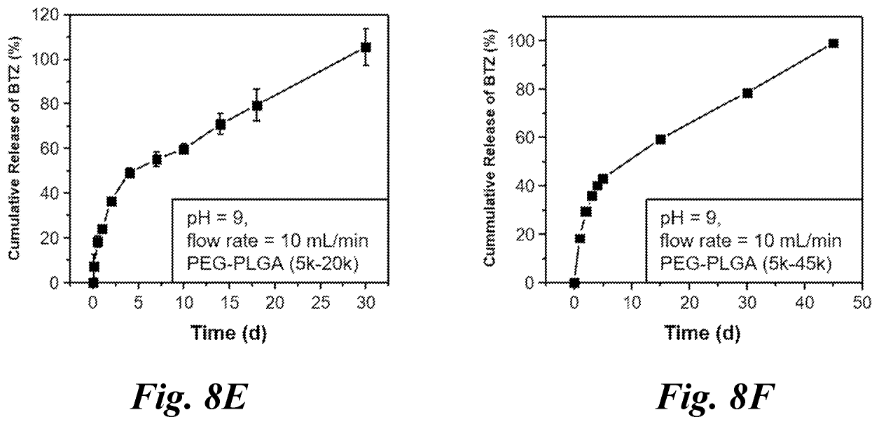

Nanoparticles were synthesized through the sequential FNC/FNP process as shown in FIG. 1. The binding efficiency between BTZ and TA in the FNC step for forming BTZ-TA complex depends on the pH of TA solution: a higher pH increases negative charge density of TA, facilitates the coordination complex formation with the boronic acid residues of BTZ, thus increasing stability of BTZ-TA complex. As the pH increased from 5 to 9, the release duration of the encapsulated BTZ increased from about 5 to 10 days (FIG. 8A, FIG. 8B, and FIG. 8C).

In the FNC/FNP process of nanoparticle formation, the flow rate plays a significant role in generating efficient mixing and improving uniformity of the NPs. Increasing the flow rate from 1 mL/min to 10 mL/min in the FNC step for TA/BTZ complexation effectively extended the releasing duration from 5 to 10 days. By combining the effects of higher pH (pH=9) and higher flow rate condition (10 mL/min), the prepared BTZ NPs yielded a substantially extended release duration to about 30 days (FIG. 8E) with a high encapsulation efficiency of 86.2%.

In the third step of BTZ nanoparticle formation, the polymer structure and characteristics, such as the molecular weight of PEG-b-PLGA, also influence the BTZ release profile. Increasing the molecular weight of the PLGA block from 20 KDa to 45 kDa extended the releasing duration from 30 days to 45 days (FIG. 8F). The stronger hydrophobic interaction due to the increase of PLGA molecular weight yielded slower degradation, slower dissociation of BTZ/TA/OVA complexes, and thus a slower release rate of BTZ.

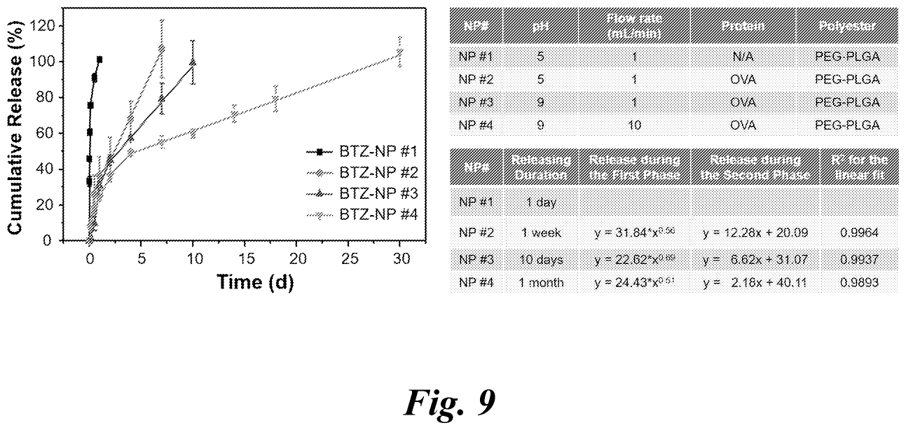

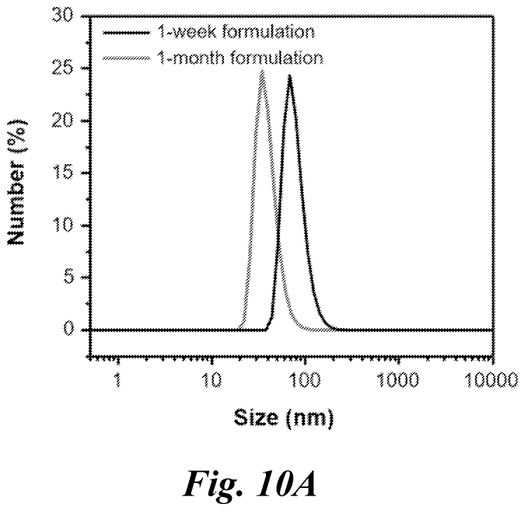

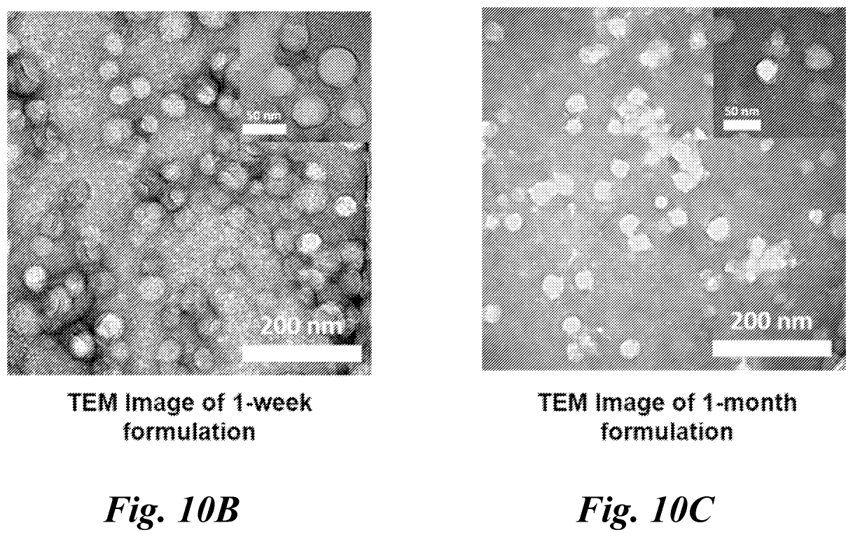

FIG. 9 shows a set of release profiles of four representative NP formulations with distinctive release rates and durations. A 1-week formulation was prepared at pH 5 and a flow rate of 1 mL/min; and a 1-month formulation was obtained at a higher pH and a high flow rate (pH=9, 10 mL/min). Both the 1-week formulation and the 1-month formulation were characterized using the dynamic light scattering (DLS) measurements and transmission electronic microscopy (TEM) as shown in FIG. 10.

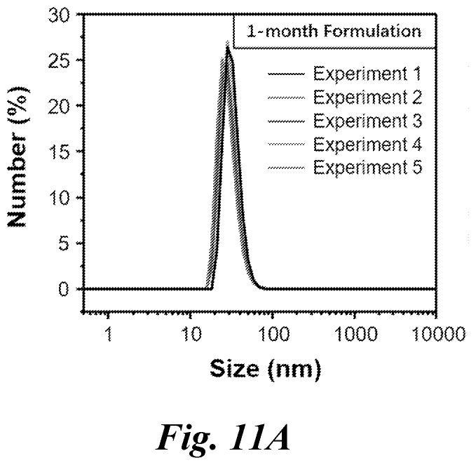

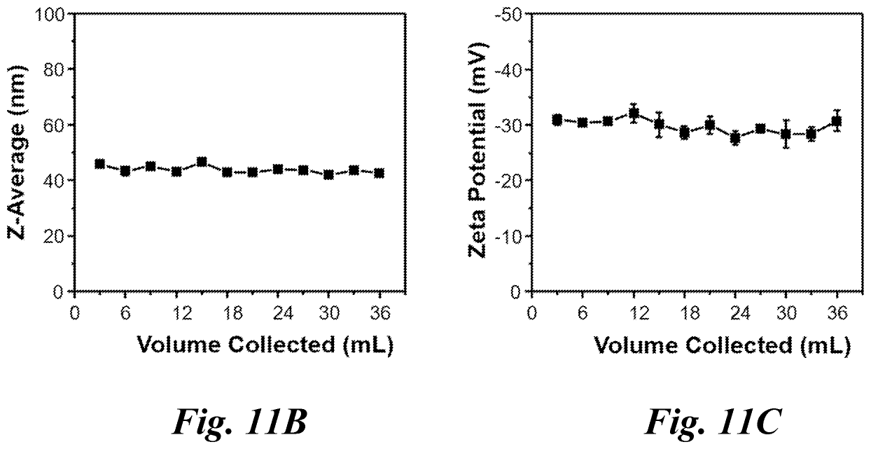

This NP production process has good reproducibility and scalability as validated through samples prepared in multiple batches (FIG. 11A) and sample collected at different time points during a single batch production. As shown in FIG. 11B, C, all of these samples showed high degree of consistency in terms of both nanoparticle size and surface charge.

Accordingly, in some embodiments, the presently disclosed subject matter provides a nanoparticle comprising bortezomib encapsulated in a non-water-soluble polymer matrix in a form of a bortezomib-tannic acid complex.

In certain embodiments, the bortezomib-tannic acid complex is bonded and stabilized to one or more proteins or peptides via hydrogen bond formation. In particular embodiments, the weight percentage of the one or more protein or peptides is the range from about 5 w/w % to about 20 w/w %. In particular embodiments, the one or more proteins or peptides has a molecular weight in the range of about 1 kDa to about 160 kDa, including about 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, and 160 kDa. In some embodiments, the one or more proteins or peptides has a molecular weight between 1 kDa to 160 kDa, 5 kDa to 140 kDa, or 30 kDa to 120 kDa. In more particular embodiments, the one or more proteins comprises a serum albumin. In yet more particular embodiments, the serum albumin is selected from the group consisting of recombinant human serum albumin, bovine serum albumin, mouse serum albumin, ovalbumin, collagen, gelatin, and protamine.

An example of a suitable protein used in the present invention is ovalbumin (OVA). An example of peptide could be a non-immunogenic sequence comprising 10 to 30 amino acid residues.

A suitable non-water-soluble polymer is one or more biodegradable polyesters or copolymers of biodegradable polyesters and other biodegradable polymers such as poly(amino acid)s, polycarbonates, and polyphosphoesters.

In certain embodiments, the non-water-soluble polymer matrix comprises one or more biodegradable polyesters. In particular embodiments, the non-water-soluble polymer matrix comprises one or more polymers selected from the group including poly(D-lactic acid) (PDLA), poly(L-lactic acid) (PLLA), poly(D,L-lactic acid) (PDLLA), poly(glycolic acid) (PGA), polycaprolactone (PCL), and their copolymers such as poly(lactic acid-co-glycolic acid) (PLGA), poly(caprolactone-co-glycolic acid) (PCLGA), and the like.

In more particular embodiments, the non-water-soluble polymer matrix comprises one or more block copolymers of polyester with poly(ethylene glycol) (PEG), selected from the group including poly(ethylene glycol)-b-poly(D-lactic acid) (PEG-b-PDLA), poly(ethylene glycol)-b-poly(L-lactic acid) (PEG-b-PLLA), poly(ethylene glycol)-b-poly(D,L-lactic acid) (PEG-b-PDLLA), poly(ethylene glycol)-b-poly(glycolic acid) (PEG-b-PGA), poly(ethylene glycol)-b-polycaprolactone (PEG-b-PCL), poly(ethylene glycol)-b-poly(lactic acid-co-glycolic acid) (PEG-b-PLGA), poly(ethylene glycol)-b-poly(caprolactone-co-glycolic acid) (PEG-b-PCLGA), and the like. In even more particular embodiments, the non-water-soluble polymer comprises poly(ethylene glycol)-b-poly(lactic acid-co-glycolic acid) (PEG-b-PLGA).

In certain embodiments, the bortezomib is released from the nanoparticle over a period of time ranging from about 2 to about 60 days in vitro, including about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 days.

In some embodiments, the nanoparticle further comprises one or more additional chemotherapy agents.

A “chemotherapeutic agent” is used to connote a compound or composition that is administered in the treatment of cancer. Chemotherapeutic agents contemplated for use in combination with the presently disclosed nanoparticles, or a pharmaceutical composition thereof include, but are not limited to, alkylating agents, such as thiotepa and cyclophosphamide; alkyl sulfonates, such as busulfan, improsulfan and piposulfan; aziridines, such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas, such as carmustine, chlorozotocin, fotemustine, lomustine, nimustine, ranimustine;

antibiotics, such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, such as methotrexate and 5-fluorouracil (5-FU); folic acid analogues, such as denopterin, methotrexate, pteropterin, trimetrexate; purine analogs, such as fludarabine, 6-mercaptopurine, thiamiprine, thioguanine; pyrimidine analogs, such as ancitabine, azacitidine, 6-azauridine, carmofur, cytosine arabinoside, dideoxyuridine, doxifluridine, enocitabine, floxuridine, 5-FU; androgens, such as calusterone, dromostanolone propionate, epitiostanol, mepitiostane, testolactone; anti-adrenals, such as aminoglutethimide, mitotane, trilostane; folic acid replenishers, such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2,2-trichlorotriethylamine; urethan; vindesine; dacarbazine; mannomustine; mitobronitol; mitolactol; pipobroman; gacytosine; arabinoside (Ara-C); taxoids, e.g., paclitaxel and docetaxel; chlorambucil; gemcitabine; 6-thioguanine; mercaptopurine; platinum analogs, such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above.

Chemotherapeutic agents also include anti-hormonal agents that act to regulate or inhibit hormone action on tumors, such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens, such as flutamide, nilutamide, bicalutamide, leuprolide, and goserelin; and pharmaceutically acceptable salts, acids or derivatives of any of the above.

In some embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these.

In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain alternative embodiments, the antimitotic agent comprises a vinca alkaloid, such as vincristine, binblastine, vinorelbine, or vindesine, or pharmaceutically acceptable salts, acids, or derivatives thereof.

In other embodiments, the presently disclosed subject matter provides a method for making a nanoparticle, the method comprising: (a) mixing bortezomib (BTZ) and tannic acid (TA) to form a BTZ/TA complex; (b) mixing a protein with the TA/BTZ complex forming a BTZ/TA/protein complex; (c) mixing a non-water soluble polymer with the BTZ/TA/protein complex; and (d) forming a nanoparticle.

In certain embodiments, the tannic acid is in an aqueous solution and has a concentration ranging from about 1 mg/mL to about 20 mg/mL, including about 1, 2, 3, 4, 5, 6, 7, 8, 9, 10, 11, 12, 13, 14, 15, 16, 17, 18, 19, and 20 mg/mL. In certain embodiments, the BTZ is in a solution comprising 0-10% acetonitrile/2-10% dimethyl sulfoxide/80-96% water. In certain embodiments, the tannic acid and the bortezomib are mixed by simultaneously injecting tannic acid and bortezomib into a 2-inlet confined impinging jet (CIJ) mixer at a flow rate in the range of about 0.2 to about 25 mL/min, including about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, and 25 mL/min, to form the TA/BTZ complex.

In particular embodiments, the BTZ/TA complex and the protein are mixed in an aqueous suspension by simultaneously injecting the BTZ/TA complex and the protein into a second 2-inlet CIJ mixer at a flow rate of about 0.2 to about 25 mL/min, including about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, and 25 mL/min, to form the protein complex.

In certain embodiments, the protein comprises a serum albumin. In yet more particular embodiments, the serum albumin is selected from the group consisting of recombinant human serum albumin, bovine serum albumin, mouse serum albumin, ovalbumin, collagen, gelatin, and protamine.

In certain embodiments, the protein complex and the non-water-soluble polymer are mixed in DMSO/acetonitrile mixture at a volume ratio of about 0 to about 1, including about 0, 0.1, 0.2, 0.3, 0.4, 0.5, 0.6, 0.7, 0.8, 0.9, and 1.0, by simultaneously injecting the BTZ/TA/protein complex and the non-water-soluble polymer into a 3-inlet CIJ mixer at a flow rate of about 0.1 to about 25 mL/min, including about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, and 25 mL/min, thereby forming the nanoparticles.

In certain embodiments, the non-water-soluble polymer matrix comprises one or more biodegradable polyesters. In particular embodiments, the non-water-soluble polymer matrix comprises one or more polymers selected from the group including poly(D-lactic acid) (PDLA), poly(L-lactic acid) (PLLA), poly(D,L-lactic acid) (PDLLA), poly(glycolic acid) (PGA), polycaprolactone (PCL), and their copolymers such as poly(lactic acid-co-glycolic acid) (PLGA), poly(caprolactone-co-glycolic acid) (PCLGA), and the like. In more particular embodiments, the non-water-soluble polymer matrix comprises one or more block copolymers of polyester with poly(ethylene glycol) (PEG), selected from the group including poly(ethylene glycol)-b-poly(D-lactic acid) (PEG-b-PDLA), poly(ethylene glycol)-b-poly(L-lactic acid) (PEG-b-PLLA), poly(ethylene glycol)-b-poly(D,L-lactic acid) (PEG-b-PDLLA), poly(ethylene glycol)-b-poly(glycolic acid) (PEG-b-PGA), poly(ethylene glycol)-b-polycaprolactone (PEG-b-PCL), poly(ethylene glycol)-b-poly(lactic acid-co-glycolic acid) (PEG-b-PLGA), poly(ethylene glycol)-b-poly(caprolactone-co-glycolic acid) (PEG-b-PCLGA), and the like. In even more particular embodiments, the non-water-soluble polymer comprises poly(ethylene glycol)-b-poly(lactic acid-co-glycolic acid) (PEG-b-PLGA).

In some embodiments, the nanoparticle further undergoes dialysis for a period of time ranging from about 6 to 12 hours, including 6, 7, 8, 9, 10, 11, and 12 hours.

In other embodiments, the presently disclosed subject matter provides a method for treating liver cancer in a subject in need of treatment thereof, the method comprising delivering one or more of the presently disclosed nanoparticles to the subject by intratumor injection to treat the liver cancer.

In certain embodiments, the intratumor injection is in an artery forming an intratumor injection tract and further comprises the step of blocking off the artery(ies) that feed the liver cancer after the delivery of the nanoparticle. In particular embodiments, the blocking occurs by transarterial embolization.

In certain embodiments, the method further comprises plugging an intratumor injection tract. In certain embodiments, the one or more nanoparticles are delivered by catheter-based intra-tumoral intra-vascular delivery. In particular embodiments, the catheter-based intratumoral intra-vascular delivery is followed by an embolization blockage to achieve a local retention and release of bortezomib.

The presently disclosed nanoparticles also can be administered in combination with one or more therapeutic agents, such as the chemotherapeutic agents provided hereinabove.

The term “combination” is used in its broadest sense and means that a subject is administered at least two agents, more particularly a presently disclosed nanoparticle and at least one chemotherapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the nanoparticles described herein can be administered alone or in combination with adjuvants that enhance stability of the nanoparticles, alone or in combination with one or more chemotherapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the presently disclosed nanoparticles and at least one additional therapeutic agent can be varied so long as the beneficial effects of the combination of these agents are achieved. Accordingly, the phrase “in combination with” refers to the administration of a presently disclosed nanoparticle and at least one additional therapeutic agent either simultaneously, sequentially, or a combination thereof. Therefore, a subject administered a combination of a presently disclosed nanoparticle and at least one additional therapeutic agent can receive a presently disclosed nanoparticle and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the presently disclosed nanoparticle and at least one additional therapeutic agent are administered simultaneously, they can be administered to the subject as separate pharmaceutical compositions, each comprising either a presently disclosed nanoparticle or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a presently disclosed nanoparticle and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index(SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### Pharmaceutical Preparations

Pharmaceutical compositions of the present invention comprise an effective amount of one or more nanoparticles of the present including those comprising BTZ, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one nanoparticle of the present invention or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The nanoparticles of the present invention may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

One or more nanoparticles of the present invention may be formulated into a composition in a free base, neutral or salt form. Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

In accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, and the like.

In further embodiments, the present invention may concern the use of a pharmaceutical lipid vehicle compositions that include one or more nanoparticles of the present invention, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, one or more nanoparticle of the present invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will determine the concentration of active ingredient(s) in a composition and appropriate dose(s) for the individual subject.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In one embodiment of the present disclosure, one or more nanoparticles are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, one or more nanoparticles of the present invention may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035−1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Pharmaceutical Compositions and Formulations

In other preferred embodiments of the invention, one or more nanoparticles of the present invention may be formulated for administration via various miscellaneous routes, for example, topical (i.e., transdermal) administration, mucosal administration (intranasal, vaginal, etc.) and/or inhalation. Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits Comprising the Presently Disclosed Nanoparticles

Any of the compositions described herein may be comprised in a kit. In a non-limiting example, one or more of the nanoparticles of the present invention (for example, those including BTZ) may be comprised in a kit.

The kits may comprise a suitably aliquoted one or more nanoparticles of the present invention and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing one or more nanoparticles of the present invention and any other reagent containers in close confinement for commercial sale. Such containers may include injection or blow-molded plastic containers into which the desired vials are retained. When the components of the kit are provided in one and/or more liquid solutions, the liquid solution is an aqueous solution, with a sterile aqueous solution being particularly preferred. One or more nanoparticle composition(s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

The components of the kit, however, may be provided as dried powder(s). When reagents and/or components are provided as a dry powder, the powder can be reconstituted by the addition of a suitable solvent. It is envisioned that the solvent may also be provided in another container means.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function.

By “agent” is meant any small molecule chemical compound such as BTZ, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. “

By “analog” is meant a molecule that is not identical, but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “bortezomib” or “BTZ” is meant a drug that may be sold under the brand Velcade among others, is an anti-cancer medication used to treat multiple myeloma and mantle cell lymphoma and has the following chemical structure of Formula I:

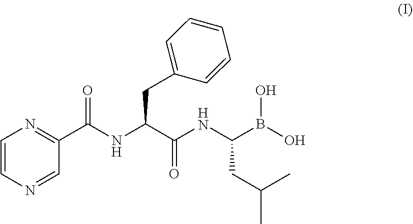 [SMILES: CC(C)C[C@H](NC(=O)[C@H](Cc1ccccc1)NC(=O)c1cnccn1)B(O)O]

or a pharmaceutically acceptable salt, solvate, or stereoisomer thereof.

By “disease” is meant any condition or disorder that damages or interferes with the normal function of a cell, tissue, or organ. An example is cancer.

By “bortezomib-tannic acid complex” is meant complexes of BTZ with tannic acid.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “express” refers to the ability of a gene to express the gene product including for example its corresponding mRNA or protein sequence (s).

The term, “obtaining” as in “obtaining an agent” includes synthesizing, purchasing, or otherwise acquiring the agent.

By “prevent,” “preventing,” “prevention,” or “prophylactic treatment” and the like is meant reducing the probability of developing a disorder or condition in a subject, who does not have, but is at risk of or susceptible to developing a disorder or condition.

By “ranges” is meant to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration of at least 10%, 25%, 50%, 75%, or 100%.

By “reference” is meant a standard or control conditions such as a sample (human cells) or a subject that is a free, or substantially free, of an agent such as a nanoparticle containing BTZ of the present invention.

By “subject” is meant to refer to any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

By “treat,” treating,” or “treatment,” is meant reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Examples

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

Device and Materials. Ultrafiltration tubes were purchased from Sartorius NE-4000 Programmable Multi-Channel Syringe Pump was purchased from New Era Company. Thermo Scientific™ Hypersil™ BDS C18 HPLC Columns (4.6 mm×250 mm) was purchased from Thermo Fisher Scientific. PEG5 KDa-b-PLGA20 KDa and PEG5 KDa-b-PLGA45 KDa (Poly SciTech®) were used as representative hydrophobic biodegradable polymers for nanoparticle preparation. Tannic acid (TA) was obtained from Sigma-Aldrich; and ovalbumin (OVA) purchased from Sigma-Aldrich (U.S.) was used as a surrogate carrier protein or peptide. HPLC grade water, HPLC grade acetonitrile were purchased from Thermo Fisher Scientific (U.S). Dialysis tubes were purchased from Spectrum Labs (U.S). Bortezornib was purchased from LC Laboratories (U.S.). Cy7.5 carboxylic acid was purchased from Lumiprobe (U.S.).

Set-up and General Procedure for the FNC/FNP Method. The working solutions were prepared as follows: (1) BTZ was dissolved in a solvent composed of dimethyl sulfoxide: acetonitrile: distilled water (5:5:90, v/v) at a concentration of 1 mg/mL; (2) Tannic acid was dissolved in distilled water (pH=5) at a concentration of 0.5, 1, 2, 4, 8, 12 and 16 mg/mL; (3) OVA was dissolved in distilled water (pH=7.5) at a concentration of 1 mg/mL; and (4) PEG5 KDa-b-PLGA20 KDa was dissolved in pure acetonitrile at a concentration of 3, 5, 7 mg/mL. Controlled by digital syringe pumps (New ERA, NE-4000, USA), solutions of BTZ and TA were simultaneously injected into the two-inlet confined impingement jets (CIJ) mixer under a flow rate of 1, 3, 5 mL/min to form the BTZ-TA complex (Step 1). Then the BTZ/TA complex and OVA were simultaneously injected into the two-inlet CIJ mixer under a flow rate of 1, 8, 15 mL/min to coat the complex with a layer of OVA (Step 2). Finally, the BTZ/TA complex or OVA coated BTZ-TA complex, water and PEG-PLGA were injected into a 3-inlet CIJ mixer for the FNP step under a flow rate of 15 mL/min (Step 3, FIG. 1). The first 1 mL of the efflux mixture may contain less well-defined nanoparticles during the initial establishment of the steady flow, and therefore was discarded. The resulted nanoparticles were dialyzed in a 100-kDa dialysis tube for 6 hours to remove free components and organic solvents before use.

Preparation of BTZ-NPs with an extended BTZ release duration. The working solutions were prepared as follows: (1) BTZ was dissolved in a solvent composed of dimethyl sulfoxide: distilled water (10:90, v/v) at a concentration of 1 mg/mL; (2) Tannic acid was dissolved in distilled water (pH=5, 7, or 9) at a concentration of 4 mg/mL; (3) OVA was dissolved in distilled water (pH=7.5) at a concentration of 1 mg/mL; and (4) PEG5 KDa-b-PLGA20 KDa or PEG5 KDa-b-PLGA45 KDa was dissolved in pure acetonitrile at a concentration of 7 mg/mL. Controlled by digital syringe pumps (New ERA, NE-4000, USA), solutions of BTZ and TA were simultaneously injected into a two-inlet confined impingement jets (CIJ) mixer under a flow rate of 10 mL/min to form the BTZ-TA complex (Step 1). The BTZ/TA complex and OVA were then simultaneously injected into another two-inlet CIJ mixer under a flow rate of 15 mL/min to form BTZ/TA/OVA complexes (Step 2). In the third step, the BTZ/TA/OVA complex suspension, water and PEG-b-PLGA solution were injected into a 3-inlet CIJ mixer under a flow rate of 10 mL/min (Step 3, FIG. 1). The first 1 mL of the efflux mixture was discarded, and the collected nanoparticles were dialyzed in a 100-kDa dialysis tube against water for 6 hours to remove free BTZ and organic solvents before use.

Characterization of Nanoparticles. The dynamic light scattering (DLS) measurements were performed using a Malvern Zetasizer Nano ZS at 25° C. to determine hydrodynamic size, and polydispersity index (PDI). The morphology of the nanoparticles was assessed by transmission electron microscopy (TEM) on FEI Tecnai 12 (USA). The encapsulation efficiency (EE) of BTZ was assessed by measuring unencapsulated BTZ in the supernatant. Briefly, the nanoparticle suspension was filtered using an ultrafiltration tube (MWCO 50 kDa) at 300×g for 20 min at room temperature. The concentrations of BTZ in the filtrate were determined by HPLC at 280 nm as the unencapsulated BTZ to calculate the final encapsulation efficiency.

$$EE ( \% ) = ( 1 - \frac{Amount of unencapsulated BTZ}{Total amount of BTZ added} ) \times 100 \% \qquad ( 1 )$$

In vitro release study of BTZ-loaded nanoparticles. Nanoparticle samples (1 mL) were pipetted into dialysis tubes (MWCO 100 kDa) and incubated in 9 mL of PBS with 0.1% v/v tween-20 at 37° C. on a shaker at 100 rpm. Release samples (200 μL) were collected at specific time points of 8, 16, 32, 56, 80, 104 and 128 h; and the medium was replenished with fresh PBS to maintain a constant volume. The concentration of released BTZ in medium was measured by HPLC. The HPLC analysis of BTZ was conducted with mobile phase composed of acetonitrile: water (60/40, v/v), maintaining a flow rate of 1 mL/min. The UV detector was set at 280 nm for absorption and fitting to a standard curve, and then linked to computer software for data analysis.

MTT assay. MDA cells (epithelial breast cancer cells, used as a prototype epithelial cancer cell line system) were seeded in a 96-well plate at a density of 10,000 cells per well, and incubated with PBS, free BTZ (25, 18.75, 12.5, 6.25 μg/mL) and NP-3 (12.5 and 6.25 μg/mL). After 4 hours, medium was removed, and wells were washed with PBS. The MTT reagent was dissolved in medium to a final concentration of 0.5 mg/mL in each well, followed by incubation for 4 hours at 37° C. After removing the MTT-contained medium, DMSO solution (150 μL/well) was added, and the plate was shaken on a microplate stirrer for 10 minutes to dissolve the crystal. The OD value of each well was detected by a microplate reader (detection wavelength 570 nm).

In vivo therapeutic effect of BTZ-loaded nanoparticles. The therapeutic effect of BTZ-loaded nanoparticles was assessed using patient derived xenograft (PDX) model. Briefly, fresh human tumor tissue was harvested from HCC patients, then cut in small pieces (3\hat{ }3 mm³) and implanted subcutaneously in the right flank of NSG mice. Once tumors grew, a similar process (harvesting tumors from mice, mincing and inserting in new mice) was implemented. Mice at the 5th passage of PDX were utilized in this experiment. After implantation subcutaneously, the tumor is allowed to grow to approximately 1,000 mm³. Next, mice were randomized to blank nanoparticles and BTZ-loaded nanoparticles with an equivalence of 2.2 mg/kg BTZ. The tumor sizes of mice in different groups were measured through two weeks and were taken as the reflection of therapeutic effect of different formulations.

In vivo retention study using Cy 7.5 labelled nanoparticles. Cy 7.5 carboxylic acid was used to labelled PEG-PLGA through Michael addition as previously described. Howard et al., 2019. BTZ-loaded nanoparticle was prepared using the same formulation of NP-3 with dye-labelled polymer. After intra-tumor injection of 100 μL of NP-3 suspension, the biodistribution of Cy 7.5-nanoparticle was revealed by near infrared imaging using the IVIS system (Progama, US) with ex 780 nm and em 810 nm at 0, 1, 3, 5, 7, 10 d post-administration.

Dose escalation test on animal survival following local subcutaneous injection of the 1-month BTZ nanoparticle (BTZ NP) formulation in comparison with free BTZ injection via intratumor injection.

Table 1 demonstrates drastically different toxicity and survival of mice receiving free BTZ or BTZ-NPs at different dose levels. The recommended dose in patients is 1.3 mg/m². This dose is also the maximum tolerated dose in patients. In our rodent model, we found that a dose of 1.5 mg/m² is lethal too the experimental animals. All 5 mice injected intratumorally with this dose of free BTZ died at day 2 and 3. One of the essential features of BTZ-NP is the ability to release active BTZ gradually. The ability of BTZ-NP to release BTZ slowly by subcutaneous injections was evaluated with increasing doses of BTZ-NP. As shown, a dose of 108 mg/m² could be injected without any death in rodents. This dose is approximately 80-fold higher than the dose of the free drug that is sufficient to kill rodents.

TABLE 1
Dose escalation test on animal survival following a single local
subcutaneous injection of the 1-month BTZ nanoparticle (BTZ NP) formulation in
comparison with free BTZ via a single intratumor injection.

| Treatment | Injection route | BTZ dose | | Equivalent BTZ dose | | Survival |
|---|---|---|---|---|---|---|
| 1 mg/kg | Intra-tumor | 0.125 | mg/mL | 12.5 | μg/dose | 0/5 |
| free BTZ | injection | 100 | μL | 1.5 | mg/m² | (died on day 2-3) |
| | | | | | | liver failure |
| 1 mg/kg | Subcutaneous | 0.6 | mg/mL, | 3.6 | mg/m² | 3/3 |
| BTZ NP | injection of | 50 | μL | | | |
| 1.5 mg/kg | 1-month release | 0.6 | mg/mL, | 5.4 | mg/m² | 3/3 |
| BTZ NP | formulation of | 75 | μL | | | |
| 2 mg/kg | BTZ NP | 0.6 | mg/mL, | 7.2 | mg/m² | 9/9 |
| BTZ NP | in heathy | 100 | μL | | | |
| 3 mg/kg | wild type mice | 0.6 | mg/mL, | 10.8 | mg/m² | 6/6 |
| BTZ NP | | 150 | μL | | | |
| 4 mg/kg | | 0.6 | mg/mL, | 14.4 | mg/m² | 4/4 |
| BTZ NP | | 200 | μL | | | |
| 6 mg/kg | | 0.6 | mg/mL, | 21.6 | mg/m² | 4/4 |
| BTZ NP | | 300 | μL | | | |
| 20 mg/kg | | 0.6 | mg/mL, | 72 | mg/m² | 4/4 |
| BTZ NP | | 1 | mL | | | |
| 30 mg/kg | | 0.6 | mg/mL, | 108 | mg/m² | 1/1 |

| | | |
|---|---|---|
| BTZ NP | 1.5 | mL |

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Llovet, J. M.; Zucman-Rossi, J.; Pikarsky, E.; Sangro, B.; Schwartz, M.; Sherman, M.; Gores, G., Hepatocellular carcinoma. Nature Reviews Disease Primers 2016, 2 (1), 16018.

Shen, J.; Song, G.; An, M.; Li, X.; Wu, N.; Ruan, K.; Hu, J.; Hu, R., The use of hollow mesoporous silica nanospheres to encapsulate bortezomib and improve efficacy for non-small cell lung cancer therapy. Biomaterials 2014, 35 (1), 316-326.

Ashley, J. D.; Stefanick, J. F.; Schroeder, V. A.; Suckow, M. A.; Kiziltepe, T.; Bilgicer, B., Liposomal Bortezomib Nanoparticles via Boronic Ester Prodrug Formulation for Improved Therapeutic Efficacy in Vivo. Journal of Medicinal Chemistry 2014, 57 (12), 5282-5292.

Shen, S.; Du, X.-J.; Liu, J.; Sun, R.; Zhu, Y.-H.; Wang, J., Delivery of bortezomib with nanoparticles for basal-like triple-negative breast cancer therapy. Journal of Controlled Release 2015, 208, 14-24.

Hu, Y.; He, L.; Ma, W.; Chen, L., Reduced graphene oxide-based bortezomib delivery system for photothermal chemotherapy with enhanced therapeutic efficacy. Polymer International 2018, 67 (12), 1648-1654.

He, Z.; Hu, Y.; Gui, Z.; Zhou, Y.; Nie, T.; Zhu, J.; Liu, Z.; Chen, K.; Liu, L.; Leong, K. W.; Cao, P.; Chen, Y.; Mao, H.-Q., Sustained release of exendin-4 from tannic acid/Fe (III) nanoparticles prolongs blood glycemic control in a mouse model of type II diabetes. Journal of Controlled Release 2019, 301, 119-128.

He, Z.; Nie, T.; Hu, Y.; Zhou, Y.; Zhu, J.; Liu, Z.; Liu, L.; Leong, K. W.; Chen, Y.; Mao, H.-Q., A polyphenol-metal nanoparticle platform for tunable release of liraglutide to improve blood glycemic control and reduce cardiovascular complications in a mouse model of type II diabetes. Journal of Controlled Release 2020, 318, 86-97.

Jin, Y.-N.; Yang, H.-C.; Huang, H.; Xu, Z.-K., Underwater superoleophobic coatings fabricated from tannic acid-decorated carbon nanotubes. RSC Advances 2015, 5 (21), 16112-16115.

Le, Z.; Chen, Y.; Han, H.; Tian, H.; Zhao, P.; Yang, C.; He, Z.; Liu, L.; Leong, K. W.; Mao, H.-Q.; Liu, Z.; Chen, Y., Hydrogen-Bonded Tannic Acid-Based Anticancer Nanoparticle for Enhancement of Oral Chemotherapy. ACS Applied Materials & Interfaces 2018, 10 (49), 42186-42197.

Nakamura, Y.; Mochida, A.; Choyke, P. L.; Kobayashi, H., Nanodrug Delivery: Is the Enhanced Permeability and Retention Effect Sufficient for Curing Cancer? Bioconjugate chemistry 2016, 27 (10), 2225−2238.

Howard, G. P.; Verma, G.; Ke, X.; Thayer, W. M.; Hamerly, T.; Baxter, V. K.; Lee, J. E.; Dinglasan, R. R.; Mao, H.-Q., Critical size limit of biodegradable nanoparticles for enhanced lymph node trafficking and paracortex penetration. Nano Research 2019, 12 (4), 837−844.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A nanoparticle comprising bortezomib encapsulated in a non-water-soluble polymer matrix in a form of a bortezomib-tannic acid complex.

2. The nanoparticle of claim 1, wherein the bortezomib-tannic acid complex is bonded and stabilized to one or more proteins or peptides via hydrogen bond formation.

3. The nanoparticle of claim 2, wherein the weight percentage of the one or more protein or peptides is the range from about 5 w/w % to about 20 w/w %.

4. The nanoparticle of claim 2, wherein the one or more proteins or peptides has a molecular weight in the range of about 1 kDa to about 160 kDa.

5. The nanoparticle of claim 2, wherein the one or more proteins comprise a serum albumin.

6. The nanoparticle of claim 5, wherein the serum albumin is selected from the group consisting of recombinant human serum albumin, bovine serum albumin, mouse serum albumin, ovalbumin, collagen, gelatin, and protamine.

7. The nanoparticle of claim 1 or 2, wherein the non-water-soluble polymer matrix comprises one or more biodegradable polyesters.

8. The nanoparticle of claim 1 or claim 2, wherein the non-water-soluble polymer matrix comprises one or more polymers selected from the group consisting of poly(D-lactic acid) (PDLA), poly(L-lactic acid) (PLLA), poly(D,L-lactic acid) (PDLLA), poly(glycolic acid) (PGA), polycaprolactone (PCL), and copolymers thereof.

9. The nanoparticle of claim 8, wherein the copolymer is selected from the group consisting of poly(lactic acid-co-glycolic acid) (PLGA) and poly(caprolactone-co-glycolic acid) (PCLGA).

10. The nanoparticle of claim 1 or claim 2, wherein the non-water-soluble polymer matrix comprises one or more block copolymers of polyester with poly(ethylene glycol) (PEG), wherein the one or more block copolymers are selected from the group consisting of poly(ethylene glycol)-b-poly(D-lactic acid) (PEG-b-PDLA), poly(ethylene glycol)-b-poly(L-lactic acid) (PEG-b-PLLA), poly(ethylene glycol)-b-poly(D,L-lactic acid) (PEG-b-PDLLA), poly(ethylene glycol)-b-poly (gly colic acid) (PEG-b-PGA), poly(ethylene glycol)-b-polycaprolactone (PEG-b-PCL), poly(ethylene glycol)-b-poly(lactic acid-co-glycolic acid) (PEG-b-PLGA), and poly(ethylene glycol)-b-poly(caprolactone-co-glycolic acid) (PEG-b-PCLGA).

11. The nanoparticle of claim 10, wherein the non-water-soluble polymer matrix comprises poly(ethylene glycol)-b-poly(lactic acid-co-glycolic acid) (PEG-b-PLGA).

12. The nanoparticle of claim 1 or 2, wherein the bortezomib is released from the nanoparticle over a period of time ranging from about 2 to about 60 days in vitro.

13. A nanoparticle of claim 1 or 2, further comprising one or more additional chemotherapy agents.

14. A method for making a nanoparticle, the method comprising:
(a) mixing bortezomib (BTZ) and tannic acid (TA) to form a BTZ/TA complex;
(b) mixing a protein with the TA/BTZ complex forming a BTZ/TA/protein complex;
(c) mixing a non-water soluble polymer with the BTZ/TA/protein complex; and
(d) forming a nanoparticle.

15. The method of claim 14, wherein the tannic acid is in an aqueous solution and has a concentration ranging from about 1 mg/mL to about 20 mg/mL.

16. The method of claim 14, wherein the BTZ is in a solution comprising 0-10% acetonitrile/2-10% dimethyl sulfoxide/80-96% water.

17. The method of claim 14, wherein the tannic acid and the bortezomib are mixed by simultaneously injecting tannic acid and bortezomib into a 2-inlet confined impinging jet (CIJ) mixer at a flow rate in the range of about 0.2 to about 25 mL/min to form the BTZ/TA complex.

18. The method of claim 14, wherein the BTZ/TA complex and the protein are mixed in an aqueous suspension by simultaneously injecting the BTZ/TA complex and the protein into a second 2-inlet CIJ mixer at a flow rate of about 0.2 to about 25 mL/min to form the protein complex.

19. The method of claim 18, wherein the protein is selected from the group consisting of recombinant human serum albumin, bovine serum albumin, mouse serum albumin, ovalbumin, collagen, gelatin, and protamine.

20. The method of claim 14, wherein the protein complex and the non-water-soluble polymer are mixed in DMSO/acetonitrile mixture at a volume ratio of about 0 to about 1 by simultaneously injecting the BTZ/TA/protein complex and the non-water-soluble polymer into a 3-inlet CIJ mixer at a flow rate of about 0.1 to about 25 mL/min, thereby forming the nanoparticles.

21. The method of claim 14, wherein the non-water-soluble polymer matrix comprises one or more biodegradable polyesters.

22. The method of claim 14, wherein the non-water-soluble polymer matrix comprises one or more polymers selected from the group consisting of poly(D-lactic acid) (PDLA), poly(L-lactic acid) (PLLA), poly(D,L-lactic acid) (PDLLA), poly(glycolic acid) (PGA), polycaprolactone (PCL), and copolymers thereof.

23. The method of claim 22, wherein the copolymer is selected from the group consisting of poly(lactic acid-co-glycolic acid) (PLGA) and poly(caprolactone-co-glycolic acid) (PCLGA).

24. The method of claim 14, wherein the non-water-soluble polymer matrix comprises one or more block copolymers of polyester with poly(ethylene glycol) (PEG), wherein the one or more block copolymers are selected from the group consisting of poly(ethylene glycol)-b-poly(D-lactic acid) (PEG-b-PDLA), poly(ethylene glycol)-b-poly(L-lactic acid) (PEG-b-PLLA), poly(ethylene glycol)-b-poly(D,L-lactic acid) (PEG-b-PDLLA), poly(ethylene glycol)-b-poly(glycolic acid) (PEG-b-PGA), poly(ethylene glycol)-b-polycaprolactone (PEG-b-PCL), poly(ethylene glycol)-b-poly(lactic acid-co-glycolic acid) (PEG-b-PLGA), and poly(ethylene glycol)-b-poly(caprolactone-co-glycolic acid) (PEG-b-PCLGA).

25. The method of claim 24, wherein the non-water-soluble polymer matrix comprises poly(ethylene glycol)-b-poly(lactic acid-co-glycolic acid) (PEG-b-PLGA).

26. A method for treating liver cancer in a subject in need of treatment thereof, the method comprising delivering one or more nanoparticles of claim 1 to the subject by intratumor injection to treat the liver cancer.

27. The method of claim 26, wherein the intratumor injection is in an artery forming an intratumor injection tract and further comprises the step of blocking off the artery(ies) that feed the liver cancer after the delivery of the nanoparticle.

28. The method of claim 27, wherein the blocking occurs by transarterial embolization.

29. The method of claim 26, further comprising plugging an intratumor injection tract.

30. The method of claim 26, wherein the one or more nanoparticles are delivered by catheter-based intra-tumoral intra-vascular delivery.

31. The method of claim 30, wherein the catheter-based intratumoral intra-vascular delivery is followed by an embolization blockage to achieve a local retention and release of bortezomib.
